(19) United States
(12) Patent Application Publication
(10) Pub. No.: US 2010/0114000 A1
(43) Pub. Date: May 6, 2010

(54) SANITARY TOOL HAVING FUNCTIONAL LIQUID MEDICINE

(76) Inventors: Mi Sun Park, Busan (KR); Il Sun Kim, Gyeonggi-do (KR)

Correspondence Address: CHRISTOPHER PAUL MITCHELL, 888 16TH ST., NW, SUITE 800, WASHINGTON, DC, 20006, US

(21) Appl. No.: 12/443,471

(22) PCT Filed: Sep. 18, 2007
(86) PCT No.: PCT/KR07/04481

§ 371 Date: Dec. 30, 2009

(30) Foreign Application Priority Data
Sep. 28, 2006 (KR) 10-2006-0094480
Sep. 29, 2006 (KR) 10-2006-0095297
Jan. 24, 2007 (KR) 10-2007-0007323

(51) Int. Cl. A61F 13/40; A47K 7/00

(52) U.S. Cl. 604/2

(57) ABSTRACT

Disclosed is a sanitary tool having functional liquid comprising an inner barrel having a liquid storage part for storing a predetermined functional liquid therein, an absorbent member mounted to the inner barrel to absorb the functional liquid stored in the liquid storage part, an outer barrel separably connected with the inner barrel, and a cleaning member mounted to the outer barrel to wipe out the functional liquid. Accordingly, the sanitary tool having functional liquid can be more conveniently stored, carried and used.

### TECHNICAL FIELD

The present invention relates to a sanitary tool having functional liquid, and more particularly to a sanitary tool having functional liquid, capable of storing functional liquid in a barrel mounted with an absorbent member at one end and flowing the liquid to the absorbent member when necessary.

### BACKGROUND ART

In general, cotton buds are widely used as a sanitary tool for disinfecting wounds, washing sensitive parts of a human body, and cleaning electronic appliances.

The cotton bud consists of a wooden or synthetic-resin stick and an absorbent member such as cotton wool attached to one or each end of the stick. Sometimes, for more effective cleaning, the cotton bud may be used with a predetermined functional liquid, for example alcohol, as applied to the absorbent member.

However, with a conventional cotton bud in this case, a user needs to dedicatedly prepare liquid having a proper function. Furthermore, there is inconvenience in use as well as in storage and carriage since the user has to apply the liquid to the absorbent member directly.

Therefore, a sanitary tool having functional liquid therein is thus developed by the present applicant, the sanitary tool capable of storing the functional liquid in a barrel mounted with an absorbent member and flowing the stored liquid out to the absorbent member when necessary during the disinfection or cleaning work, saving the necessity of separately preparing the functional liquid and the absorbent member for absorbing the functional liquid.

### DISCLOSURE OF INVENTION

### Technical Problem

Therefore, the present invention has been made in view of the above problems, and it is an object of the present invention to provide a sanitary tool having functional liquid, improved for more convenient storage, carriage and use.

### Technical Solution

In accordance with an aspect of the present invention, the above and other objects can be accomplished by the provision of a sanitary tool having functional liquid, comprising an inner barrel having a liquid storage part for storing a predetermined functional liquid therein; an absorbent member mounted to the inner barrel to absorb the functional liquid stored in the liquid storage part; an outer barrel separably connected with the inner barrel; and a cleaning member mounted to the outer barrel to wipe out the functional liquid.

The inner barrel may comprise a liquid storage part receiving the functional liquid; an outlet for the functional liquid stored in the liquid storage part to flow out therethrough; and an inlet for the air for moving the functional liquid stored in the liquid storage part to the outlet to flow in therethrough.

The outer barrel may comprise a barrel receiving part receiving an area including the inlet of the inner barrel; and a stopper formed in the barrel receiving part and contacting with and separating from the inlet to thereby open and shut the liquid storage part.

An air path may be formed between an inner circumference of the barrel receiving part and an outer circumference of the inner barrel so that the air flows through the air path to the inlet. Therefore, the external air flows into the inlet through the air path during separation of the inner barrel from the outer barrel, thereby moving the functional liquid stored in the liquid storage part to the outlet. Thus, the functional liquid can be supplied to the absorbent member.

The sanitary tool may further comprise a supporting rim protruded from the inner circumference of the barrel receiving part to support the outer circumference of the inner barrel.

Since the supporting rim may be formed to have a protrusion/groove structure, the external air can be smoothly flown through the air path formed by grooves of the supporting rim without complete separation of the inner barrel from the outer barrel. Therefore, the functional liquid in the liquid storage part can be supplied to the absorbent member.

According to another embodiment of the present invention, a male thread may be formed on a part of the outer circumference of the inner barrel, and a female thread for engagement with the male thread is formed on a part of the inner circumference of the barrel receiving part of the outer barrel. Therefore, the inner barrel can be more easily connected and separated with respect to the outer barrel.

The female thread may be formed to have a protrusion/groove structure formed along the length direction thereof. Therefore, the external air can be smoothly flown through the air path formed by grooves of the female thread without complete separation of the inner barrel from the outer barrel, and the functional liquid in the liquid storage part can be supplied to the absorbent member.

The absorbent member and the cleaning member may be implemented by cotton wool or a nib.

In accordance with another aspect of the present invention, there is provided a sanitary tool having functional liquid, comprising an inner barrel having a liquid storage part for storing a predetermined functional liquid and an outlet formed at one end thereof for the functional liquid stored in the liquid storage part to flow out therethrough; an absorbent member mounted to the outlet to absorb the functional liquid stored in the liquid storage part; a piston mounted in the inner barrel to be movable by an elastic member; and a fixing pin separably fit with the inner barrel and the piston, thereby fixing the piston to the inner barrel.

The piston may move to keep airtightness in the inner barrel.

The absorbent member may be implemented by cotton wool or a nib.

The other end of the inner barrel may be mounted with a cleaning member.

The cleaning member may be implemented by cotton wool.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view of a sanitary tool having functional liquid, according to a first embodiment of the present invention;

FIG. 2 is a longitudinal sectional view of FIG. 1;

FIG. 3 is a longitudinal sectional view of FIG. 1, showing the use process of the sanitary tool having functional liquid according to the first embodiment of the present invention;

FIG. 4 is a perspective view of a sanitary tool having functional liquid, according to, a second embodiment of the present invention;

FIG. 5 is a longitudinal sectional view of FIG. 4;

FIG. 6 is a perspective view of a sanitary tool having functional liquid according to a third embodiment of the present invention;

FIG. 7 is a longitudinal sectional view of FIG. 6;

FIG. 8 is a longitudinal sectional view of FIG. 6, showing the use process of the sanitary tool having functional liquid according to the third embodiment of the present invention;

FIG. 9 is a longitudinal sectional view of FIG. 6;

FIG. 10 is an exploded perspective view of a sanitary tool having functional liquid, according to a fourth embodiment of the present invention;

FIG. 11 is a longitudinal sectional view of FIG. 10;

FIG. 12 is a perspective view of a sanitary tool having functional liquid, according to a fifth embodiment of the present invention;

FIG. 13 is a longitudinal sectional view of FIG. 12;

FIG. 14 is a sectional view of a sanitary tool having functional liquid, according to a sixth embodiment of the present invention;

FIG. 15 is a perspective view of a sanitary tool having functional liquid, according to a seventh embodiment of the present invention;

FIG. 16 is a longitudinal sectional view of FIG. 15.

### MODE FOR THE INVENTION

Hereinafter, certain embodiments of the present invention will be described in detail with reference to the accompanying drawings.

In the description, like elements having like functions and structures will be cited by the same reference numerals throughout and explained representatively in a first embodiment while, in other embodiments, only distinctive structures thereof will be explained.

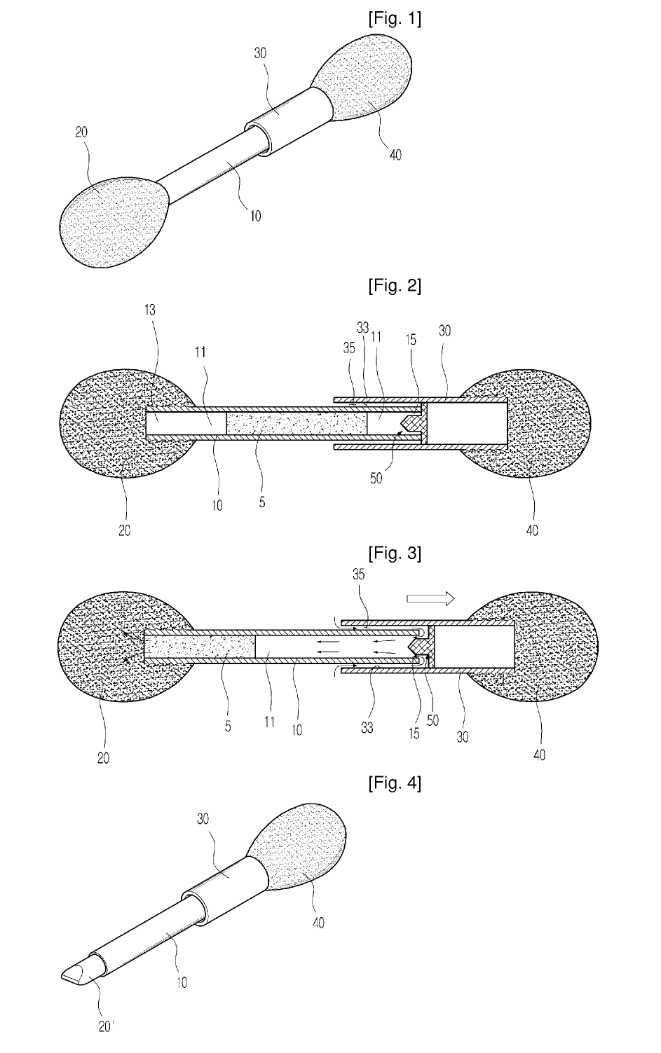

FIGS. 1 through 3 show a sanitary tool having functional liquid, according to the first embodiment of the present invention. Referring to the drawings, the sanitary tool having functional liquid comprises an inner barrel 10, an absorbent member 20, an outer barrel 30, and a cleaning member 50.

The inner barrel 10 has a hollow pipe form having a circular cross-sectional shape, and includes a liquid storage part 11 for storing a predetermined functional liquid 5 therein. An outlet 13 is formed at one end of the inner barrel 10 to flow out the stored functional liquid 5 therethrough. An inlet 15 is formed at the other end of the inner barrel 10 to guide in air for moving the stored functional liquid 5 toward the outlet 13.

The outlet 13 of the inner barrel 10 is mounted with the absorbent member 20 for absorbing the functional liquid 5 in the liquid storage part 11. In this embodiment, cotton wool having excellent absorbency is used for the absorbent member 20. Here, scratches may be formed on an outer circumference of the inner barrel 10 in a region where the absorbent member 20 is mounted, to improve coherence between the inner barrel 10 and the absorbent member 20.

The outer barrel 30 also having a hollow pipe form reciprocates in a length direction of the inner barrel 10. The outer barrel 30 comprises a barrel receiving part 33 receiving a part of the inner barrel 10 including the inlet 15, and a stopper 50 formed in the barrel receiving part 33. The stopper 50 opens and shuts the liquid storage part 11, as contacting with and separating from the inlet 15.

Inner circumference of the barrel receiving part 33 and the outer circumference of the inner barrel 10 are spaced apart from each other by a predetermined gap, thereby forming an air path 35 for guiding air into the inlet 15. Therefore, the external air flows into the inlet 15 through the air path 35 during separation of the inner barrel 10 from the outer barrel 30, so that the functional liquid 5 stored in the liquid storage part 13 can be moved to the outlet 13 and supplied to the absorbent member 20.

In addition, the cleaning member 40 is mounted around one end of the outer barrel 30, disposed opposite to the outlet 13 of the inner barrel 10, to absorb the functional liquid 5. Similarly to the absorbent member 20, the cleaning member 40 is implemented by cotton wool having excellent absorbency. Scratches may also be formed along outer circumference of the outer barrel 30 in a region where the cleaning member 40 is mounted, to improve coherence between the outer barrel 30 and the cleaning member 40. The cleaning member 40 may be used for cleaning the part the functional liquid 5 will be applied on or wiping away wrongly applied functional liquid 5.

Therefore, in the sanitary tool having functional liquid according to the embodiment of the present invention, the absorbent member 20 and the cleaning member 40 are disposed symmetrically.

The stopper 50 is formed in the outer barrel 30 and protruded by a predetermined length in an axial direction of the outer barrel 30. The stopper 50 has the same cross-sectional shape and diameter as the inner circumference of the inlet 15 so as to keep airtightness of the liquid storage part 11 when inserted in the inlet 15. As the outer barrel 30 reciprocates, the stopper 50 tightly contacts with and separates from the end of inlet 15 of the inner barrel 10, thereby opening and blocking the inlet 15. Consequently, the liquid storage part 11 is opened and shut. In addition, the stopper 50 according to the first embodiment of the present invention prevents the air guided in through the air path 35 from moving to the cleaning member 40 along the inner circumference of the outer barrel 30. Here, the stopper 50 may be formed of an elastic material such as rubber.

Hereinafter, the manufacturing process of the sanitary tool having functional liquid according to the first embodiment of the present invention will be described briefly.

First, the outer barrel 30 is mounted to one end of the inner barrel 10 forming the inlet 15 such that the stopper 50 blocks the inlet 15.

The functional liquid 5 is then injected into the liquid storage part 11 of the inner barrel 10. During this, since the liquid storage part 11 is sealed by the stopper 50 blocking the inlet 15, negative pressure is generated in the liquid storage part 11 which is disposed between the functional liquid 5 and the stopper 50.

After a predetermined amount of the functional liquid 5 is injected in the inner barrel 10, the absorbent member 20 is mounted to the outlet 13 of the inner barrel 10. Here, owing to surface tension of the functional liquid 5 and the negative pressure between the functional liquid 5 and the stopper 50, the functional liquid 5 stored in the liquid storage part 11 of the inner barrel 10 does not move to the outlet 13 of the inner barrel 10. The sanitary tool having functional liquid according to the first embodiment of the present invention is thus manufactured.

The use process of the above-structured sanitary tool having functional liquid according to the first embodiment of the present invention will now be explained.

In the state of FIG. 2 where the functional liquid 5 is injected in the liquid storage part 11 of the inner barrel 10, if the outer barrel 30 is moved to the right of the drawing as shown in FIG. 3 so that the inner barrel 10 and the outer barrel 30 are spaced away from each other, the stopper 50 is moved to the right together with the outer barrel 30. Accordingly, the inlet 15 of the inner barrel 10 is opened.

As the inlet 15 is opened, the external air flows into the inlet 15 through the air path 35, thereby increasing pressure at the inlet 15. As a result, the negative pressure between the inlet 15 and the functional liquid 5 is released and the functional liquid 5 stored in the liquid storage part 11 is moved to the outlet 13 of the inner barrel 10 to soak the absorbent member 20.

When the absorbent member 20 is soaked, a user is now able to apply the functional liquid 5 on a desired part.

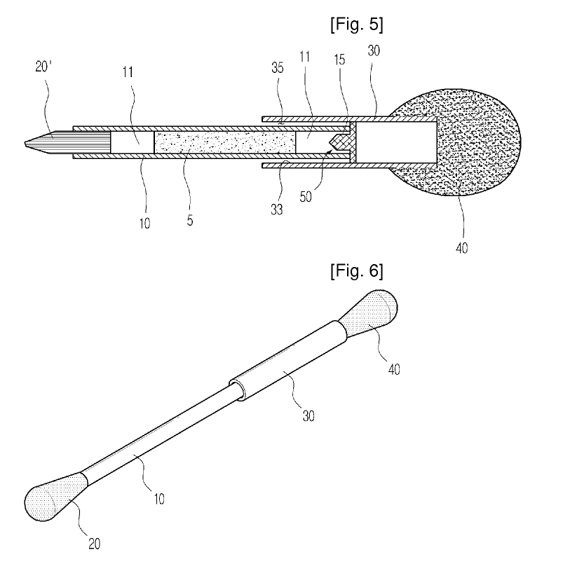

FIGS. 4 and 5 are longitudinal sectional views of a sanitary tool having functional liquid according to a second embodiment of the present invention. Differently from in the first embodiment, the sanitary tool according to the second embodiment comprises a nib as an absorbent member 20′ which is formed at the outlet 13 of the inner barrel 10.

Therefore, the sanitary tool according to the second embodiment enables more detailed application of the functional liquid 5 on the desired part, compared to the sanitary tool according to the first embodiment.

Since the process of supplying the functional liquid 5 to the absorbent member 20′ is the same as in the first embodiment, description thereof will be omitted hereinafter.

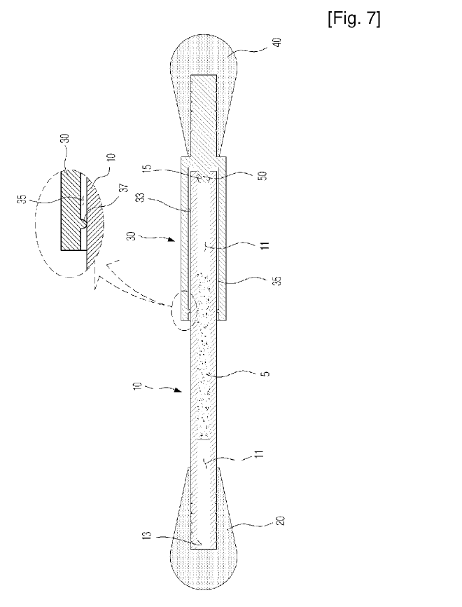

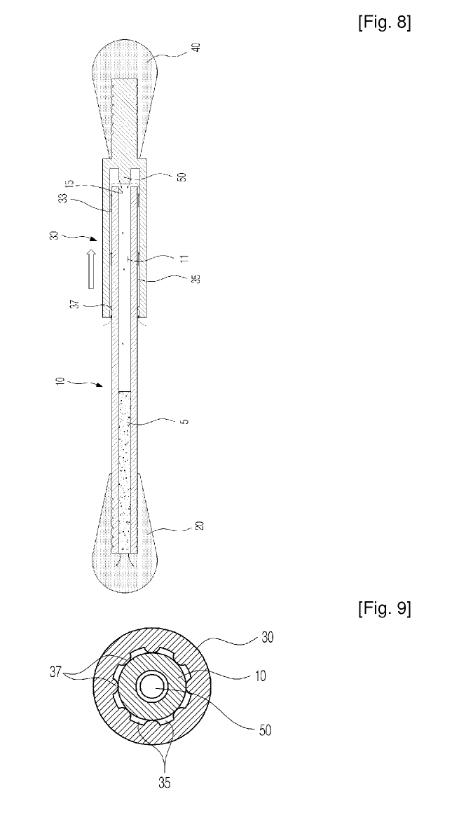

FIGS. 6 through 9 show a sanitary tool having functional liquid according to a third embodiment of the present invention. Referring to the drawings, the distinctive feature of the third embodiment from the first embodiment is a supporting rim 37 protruded on the inner circumference of the outer barrel 30 in radial directions to support the outer circumference of the inner barrel 10. More specifically, the supporting rim 37 is formed to have a protrusion/groove structure to partly support the outer circumference of the inner barrel 10.

Therefore, the external air can freely flow through the air path 35 formed by grooves of the supporting rim 37 without complete separation of the inner barrel 10 from the outer barrel 30, so that the functional liquid 5 in the liquid storage part 11 can be supplied to the absorbent member 20.

Since having been explained in the first embodiment, the process of supplying the functional liquid 5 to the absorbent member 20 will not be repeatedly explained. Although cotton wool is used for the absorbent member 20 according to the drawings of this embodiment, a nib may replace the cotton wool.

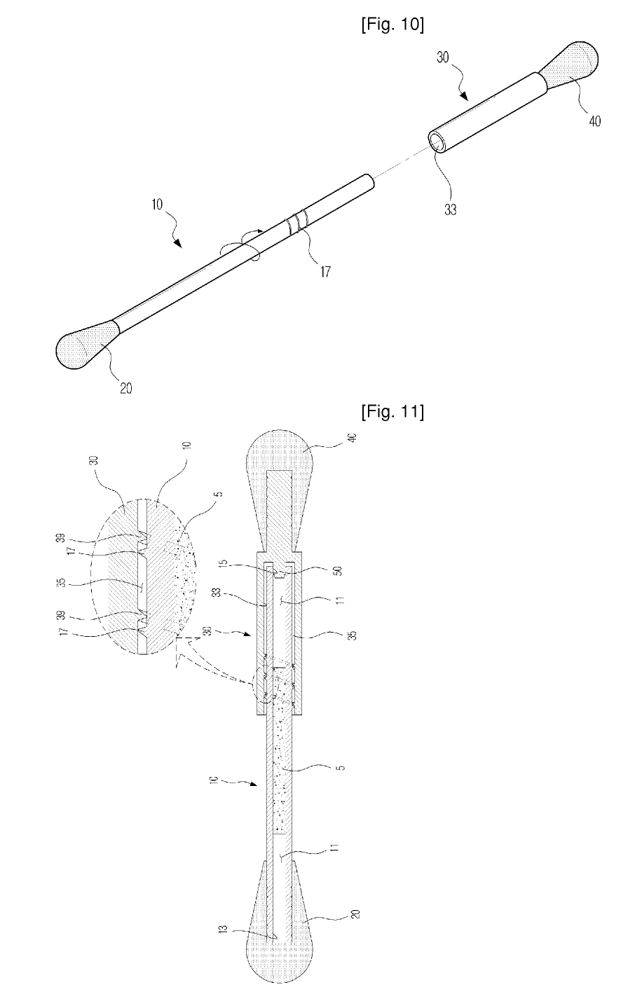

FIGS. 10 and 11 show a sanitary tool having functional liquid according to a fourth embodiment of the present invention. Although the sanitary tool of the third embodiment comprises the supporting rim 37, the sanitary tool according to the fourth embodiment comprises a male thread 17 formed on a part of the outer circumference of the inner barrel 10 and a female thread 39 formed on a part of the inner circumference of the barrel receiving part 33 of the outer barrel 30 to be engaged with the male thread 17.

The female thread 39 of the barrel receiving part 33 is formed to have a protrusion/groove structure formed along the length direction thereof. Therefore, the external air can freely flow through the air path 35 formed by grooves of the female thread 39 without complete separation of the inner barrel 10 from the outer barrel 30, so that the functional liquid 5 in the liquid storage part 11 can be supplied to the absorbent member 20.

According to the fourth embodiment of the present invention, as a result, connection and separation between the inner barrel 10 and the outer barrel 30 become easier as compared to in the third embodiment.

Since the process of supplying the functional liquid 5 to the absorbent member 20 is the same as in the first embodiment, description thereof will be omitted hereinafter. Furthermore, although cotton wool is used for the absorbent member 20 in this embodiment, the nib may replace the cotton wool.

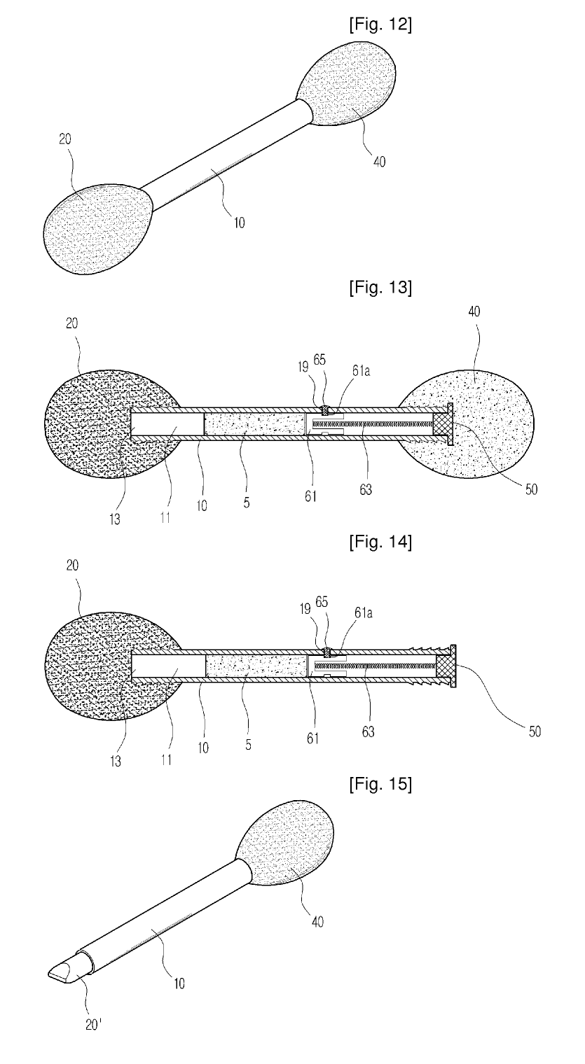

FIGS. 12 and 13 show a sanitary tool having functional liquid according to a fifth embodiment of the present invention. Differently from all the previous embodiments, the outer barrel 30 is absent from the sanitary tool according to the fifth embodiment. In addition, a piston 61 is mounted in the inner barrel 10 to be movable by an elastic member 63. The piston 61 is separably fit in the inner barrel 10 by a fixing pin 65.

Therefore, in the fifth embodiment, the outlet 13 for flowing out the functional liquid 5 from the liquid storage part 11 is formed at one end of the inner barrel 10.

The outlet 13 of the inner barrel 10 is provided with the absorbent member 20 for absorbing the functional liquid 5 in the liquid storage part 11. Cotton wool having excellent absorbency is used for the absorbent member 20 in this embodiment.

The piston 61 is mounted movably while keeping airtightness on the inner circumference of the inner barrel 10. In accordance with the movement thereof, the piston 61 pushes the functional liquid 5 in the liquid storage part 11 out to the outlet 13 of the inner barrel 10.

The stopper 50 is mounted to the other end of the inner barrel 10. The elastic member 63 is interposed between the stopper 50 and the piston 61 to supply elastic force for biasing the piston 61 toward the outlet 13 of the inner barrel 10.

The fixing pin 65 is separably fit with the piston 61 to restrict movement of the piston 61. More specifically, the fixing pin 65 is fit in a through hole 19 formed on the outer circumference of the inner barrel 10 and a fitting hole 61a formed on the piston 61. As the fixing pin 65 is released from the fitting hole 61a of the piston 61, the piston 61 is moved toward the outlet 13 thereby pushing out the functional liquid 5 to the outlet 13 of the inner barrel 10.

The cleaning member 40 may be mounted to the other end of the inner barrel 10. As well as the absorbent member 20, the cleaning member 40 is made of cotton wool having excellent absorbency.

Accordingly, in the sanitary tool having functional liquid according to the fifth embodiment of the present invention, the absorbent member 20 and the cleaning member 40 are disposed symmetrically.

Hereinafter, the use process of the above-structured sanitary tool having functional liquid according to the fifth embodiment of the present invention will be explained.

In a state where the functional liquid 5 is injected in the liquid storage part 11 of the inner barrel 10, if the fixing pin 65 is separated from the fitting hole 61a of the piston 61, the piston 61 is moved to outlet 13 by the elastic force of the elastic member 63, thereby pushing out the functional liquid 5 to the outlet 13 of the inner barrel 10.

The functional liquid 5 thus flown out to the outlet 13 soaks the absorbent member 20.

Now, the user is able to apply the functional liquid 5 on the desired part with the absorbent member 20.

FIG. 14 shows a sanitary tool having functional liquid according to a sixth embodiment of the present invention. The distinctive feature of this embodiment from the fifth embodiment is omission of the cleaning member 40 from the other end of the inner barrel 10.

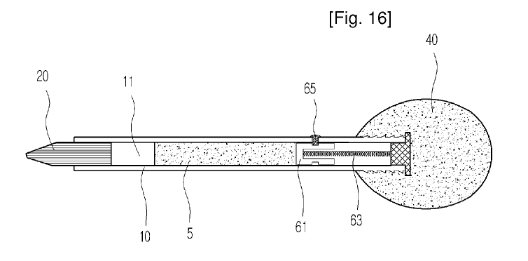

FIGS. 15 and 16 show a sanitary tool having functional liquid according to a seventh embodiment of the present invention. Differently from the fifth embodiment, the sanitary tool according to the seventh embodiment comprises a nib as the absorbent member 20′ which is formed at the outlet 13 of the inner barrel 10.

The nib-type absorbent member 20 enables more detailed application of the functional liquid 5 on the desired part in comparison with the absorbent member 20 of the fifth embodiment.

Since the process of supplying the functional liquid 5 to the absorbent member 20 is the same as in the fifth embodiment, description thereof will be omitted hereinafter.

As described above, according to the embodiments of the present invention, application of functional liquid through an absorbent member can be facilitated, saving the necessity of preparing the functional liquid and the sanitary tool separately.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A sanitary tool having functional liquid, comprising: an inner barrel having a liquid storage part for storing a predetermined functional liquid therein; an absorbent member mounted to the inner barrel to absorb the functional liquid stored in the liquid storage part; an outer barrel separably connected with the inner barrel; and a cleaning member mounted to the outer barrel to wipe out the functional liquid.

2. The sanitary tool according to claim 1, wherein the inner barrel comprises: a liquid storage part for receiving the functional liquid; an outlet for discharging the functional liquid stored in the liquid storage part; and an inlet for receiving air for moving the functional liquid stored in the liquid storage part to the outlet.

3. The sanitary tool according to claim 2, wherein the outer barrel comprises: a barrel receiving part receiving an area including the inlet of the inner barrel; and a stopper formed in the barrel receiving part and contacting with and separating from the inlet to thereby open and shut the liquid storage part.

4. The sanitary tool according to claim 3, wherein an air path is formed between an inner circumference of the barrel receiving part and an outer circumference of the inner barrel so that the air flows through the air path to the inlet.

5. The sanitary tool according to claim 4, further comprising a supporting rim protruded from the inner circumference of the barrel receiving part to support the outer circumference of the inner barrel.

6. The sanitary tool according to claim 5, wherein the supporting rim is formed to have a protrusion/groove structure.

7. The sanitary tool according to claim 4, wherein a male thread is formed on a part of the outer circumference of the inner barrel, and a female thread for engagement with the male thread is formed on a part of the inner circumference of the barrel receiving part of the outer barrel.

8. The sanitary tool according to claim 7, wherein the female thread is formed to have a protrusion/groove structure formed along the length direction thereof.

9. The sanitary tool according to claim 1, wherein the absorbent member and the cleaning member are implemented by cotton wool or a nib.

10. A sanitary tool having functional liquid, comprising: an inner barrel having a liquid storage part for storing a predetermined functional liquid and an outlet formed at one end thereof for the functional liquid stored in the liquid storage part to flow out therethrough; an absorbent member mounted to the outlet to absorb the functional liquid stored in the liquid storage part; a piston mounted in the inner barrel to be movable by an elastic member; and a fixing pin separably fit with the inner barrel and the piston, thereby fixing the piston to the inner barrel.

11. The sanitary tool according to claim 10, wherein the piston moves to keep air-tightness in the inner barrel.

12. The sanitary tool according to claim 10, wherein the absorbent member is implemented by cotton wool or a nib.

13. The sanitary tool according to any of claims 10, wherein the other end of the inner barrel is mounted with a cleaning member.

14. The sanitary tool according to claim 13, wherein the cleaning member is implemented by cotton wool.
